(19) United States
(12) Patent Application Publication
(10) Pub. No.: US 2024/0371478 A1
(43) Pub. Date: Nov. 7, 2024

(54) Method for providing dynamically customized messages in a healthcare facility

(71) Applicant: TOOLS4PATIENT, Mont-Saint-Guibert (BE)

(72) Inventors: Samuel BRANDERS, Mont-Saint-Guibert (BE); Arthur OOGHE, Mont-Saint-Guibert (BE); Guillaume BERNARD, Mont-Saint-Guibert (BE); Chantal GOSSUIN, Mont-Saint-Guibert (BE); Dominique DEMOLLE, Mont-Saint-Guibert (BE); Alvaro PEREIRA, Mont-Saint-Guibert (BE)

(73) Assignee: TOOLS4PATIENT, Mont-Saint-Guibert (BE)

(21) Appl. No.: 18/311,910

(22) Filed: May 4, 2023

(51) Int. Cl. G16H 10/20; G06F 40/40; G16H 40/20; G16H 50/20

(52) U.S. Cl. CPC G16H 10/20; G06F 40/40; G16H 40/20; G16H 50/20

(57) ABSTRACT

A computer-implemented method for providing a dynamically customized subject message and a dynamically customized health practitioner message to each subject and health practitioner in a healthcare facility, respectively, wherein each health practitioner is assigned to a plurality of subjects, the method comprising for each subject receiving subject data of the subject, receiving responses of the subject to one or more first questions of a questionnaire, generating an input dataset from the subject data and the received responses, applying a trained mathematical model on the generated input dataset to predict a profile of the subject, determining that a predetermined configuration requirement has been met, receiving healthcare facility data of the healthcare facility, and determining performance information of at least one of the plurality of subjects based on the healthcare facility data and on the subject data and predicted profile.

### FIELD OF THE INVENTION

The present invention relates to providing a dynamically customized subject message and a dynamically customized health practitioner message to each subject and health practitioner in a healthcare facility, respectively. More particularly, the present invention relates to predicting a profile of a subject.

### BACKGROUND ART

A significant portion of the world population is aging and requires increased health care as a natural result. Such healthcare treatments include medications. However, there are patients that are subject to major medical procedures such as major surgeries, transplants and the like. Therefore, the reason for increased health care can be due not just to age, but also to catastrophic events such as injuries from accidents, birth, genetic defects, and so on. Such treatment can involve heart transplants, kidney transplants, bone marrow transplants, lung transplants and the like. Each of these is a significant medical undertaking and requires significant time and energy in the recovery process to prevent complication and adverse consequences.

Every year, medical device manufacturers and pharmaceutical manufacturers undertake clinical trials to establish safety and efficacy of their products. Clinical trials are required by government regulators in major markets. Clinical trials take around six to nine years on average to complete, and may account for almost 40% of total therapy costs.

Non-compliance and non-adherence are significant challenges in the healthcare industry and can result in reduced efficacy of treatments, increased healthcare costs, and negative health outcomes for patients. These issues can be particularly problematic in clinical trials and other research studies where adherence to the study protocol is essential for maintaining the scientific validity of the study.

Therefore, there is a need for strategies to address non-compliance and non-adherence, which may include education and counseling for patients, improved medication management systems, and the development of new treatments that are more tolerable or easier to administer.

U.S. patent application Ser. No. 20/210,249962A1, titled “Systems and Methods for Promoting Patient Adherence to Treatment Regimens,” describes a method for improving patient adherence to treatment regimens. The system includes a user device that provides reminders to the patient to take their medication, and a healthcare provider device that tracks patient adherence and provides real-time feedback to the patient and their healthcare provider.

U.S. patent application Ser. No. 20/190,332292A1, titled “Method and System for Predicting Patient Adherence to Treatment Regimens,” describes a machine learning-based system for predicting patient adherence to treatment regimens. The system analyzes patient data to identify factors that may impact adherence, such as socio-demographic characteristics, comorbidities, and medication side effects, and provides personalized interventions to improve adherence.

Additionally, U.S. patent application Ser. No. 20/200,289420A1, titled “Method and System for Managing Medication Administration,” describes a medication management system that uses a mobile application to remind patients to take their medication at the correct time and provides real-time feedback to healthcare providers regarding patient adherence. The system also includes a tracking mechanism that records missed doses and identifies potential reasons for non-adherence.

Furthermore, several questionnaires including biophysical tests or virtual reality tools have already been developed and used to assess some aspects of the non-compliance or non-adherence in an individual. However, because of their stand-alone and very narrow nature, these questionnaires and biophysical tests do not provide an accurate estimation of the non-compliance or non-adherence of the individual.

Despite the huge efforts that have been made in this area, there is ongoing need for solutions to address non-compliance and non-adherence in the healthcare industry. The present invention seeks to further improve upon these solutions by providing a novel approach to addressing these issues in a more efficient and effective manner.

### SUMMARY OF THE DISCLOSURE

The present inventors have developed a method for providing a dynamically customized subject message and a dynamically customized health practitioner message to each subject and health practitioner in a healthcare facility, respectively. Surprisingly, it was found that by applying a trained mathematical model to predict a profile of a subject, a more efficient and effective way of determining whether a subject would be non-compliant or non-adherent to a treatment. Moreover, the inventors have further found that the method can reduce the number of questions asked to the subject, thereby ensuring that the subject is more engaged resulting in subject responses being more reliable. This allows for more effective treatments to subjects and more reliable studies in the healthcare industry.

Hence, according to a first aspect of the present invention, there is provided a computer-implemented method for providing a dynamically customized subject message and a dynamically customized health practitioner message to each subject and health practitioner in a healthcare facility, respectively, wherein each health practitioner is assigned to a plurality of subjects, the method comprising for each subject in the healthcare facility:
1.a. receiving, by a server, subject data of the subject,
1.b. receiving, by the server from a computing device associated with a given subject, responses of the subject to one or more first questions of a questionnaire, wherein the one or more first questions are determined, by the server, based on the subject data,
1.c. generating, by the server, an input dataset from the subject data and the received responses,
1.d. applying, by the server, a trained mathematical model on the generated input dataset to predict a profile of the subject,
1.e. dynamically generating, by the server, the customized subject message based on the predicted profile of the subject, and
1.f. transmitting in real-time, by the server, the customized subject message to the computing device associated with the subject;
1.g. upon determining, by the server, that a predetermined configuration requirement has been met:
   i. receiving, by the server, healthcare facility data of the healthcare facility;
   ii. determining, by the server, performance information of at least one of the plurality of subjects based on the healthcare facility data and on the subject data and predicted profile of said at least one of the plurality of subjects;
   iii. dynamically generating, by the server, the customized health practitioner message based on the determined performance; and
   iv. transmitting in real-time, by the server, the customized health practitioner message to a computer device associated with the health practitioner assigned to said at least one of the plurality of subjects.

The present invention relates to technical operations that require the particular technical components to perform the steps. As will be understood herein, the technical operations pertain to an improvement in the operation of technology, particularly for implementing a network including a healthcare facility. The present invention involves a server in communication with a plurality of computing devices associated with the subjects and in communication with a plurality of computer devices associated with health practitioners assigned to the subjects. Thus, the present invention transforms the nature of the interaction between the subject and a remotely located health practitioner and provides an improvement in operation of computers in a network environment for healthcare delivery, such as for implementing a clinical trial network. In embodiments according to the present invention, further comprising:
2.a. upon performing step 1.d, determining, by the server, whether the predicted profile of the subject satisfies a predetermined profile requirement, wherein when it is determined that the predicted profile does not satisfy the predetermined profile requirement, the method comprises:
2.b. determining, by the server, one or more additional questions of the questionnaire based on the predicted profile of the subject,
2.c. transmitting, by the server to the computing device, said one or more additional questions,
2.d. receiving, by the server from the computing device, responses of the subject to said one or more additional questions,
2.e. repeating, by the server, steps 2.a-2.d a predetermined number of times or until it is determined that the predicted profile satisfies the predetermined profile requirement, wherein when steps 2.a-2.d have been repeated said predetermined number of times or when it has been determined that the predicted profile satisfies the predetermined profile requirement, performing steps 1.e-1.f.

The existing methods, especially the questionnaires, are time-consuming and put a heavy burden on the subject having to undergo the testing. By determining follow-up questions based on the predicted profile of the subject, the method allows to reduce the questionnaire to a smaller and more effective number of questions. Advantageously, this ensures that the subject is more engaged, which results in subject responses being more reliable. Furthermore, this can be used to have a prediction of the subject profile without “undue” questioning of the subjects. This is particularly advantageous when the determining of the one or more additional questions is performed dynamically and the transmitting of said one or more additional questions is performed in real-time, thereby providing the subject with dynamic and real-time feedback. This is even more advantageous when said determining includes applying a trained machine learning model, such as linear regression, decision tree or a large language model, thereby ensuring that the additional questions are more relevant and effective.

According to a second aspect of the present invention, there is provided a non-transitory computer readable medium embodying computer executable instructions which when executed by a computer cause the computer to facilitate a method for providing a dynamically customized subject message and a dynamically customized and health practitioner message to each subject and health practitioner, respectively, in a healthcare facility as described herein.

According to a third aspect of the present invention, there is provided an apparatus comprising a memory embodying computer executable instructions and at least one processor, coupled to the memory, and operative by the computer executable instructions to facilitate a method for providing a dynamically customized subject message and a dynamically customized and health practitioner message to each subject and health practitioner, respectively, in a healthcare facility as described herein.

According to a fourth aspect of the present invention, there is provided a system comprising a plurality of computing devices associated with subjects, a plurality of computer devices associated with health practitioners, and an apparatus comprising a memory embodying computer executable instructions and at least one processor, coupled to the memory, and operative by the computer executable instructions to facilitate a method for providing a dynamically customized subject message and a dynamically customized and health practitioner message to each subject and health practitioner, respectively, in a healthcare facility as described herein.

Additional and alternative objects of the present invention may be understood from the following.

### BRIEF DESCRIPTION OF THE DRAWINGS

The present invention will be discussed in more detail below, with reference to the attached drawings, in which:

FIGS. 1A, 1B, 2, 4 and 5 illustrate embodiments of a method according to the present invention;

FIGS. 3 and 6 illustrate embodiments of a system according to the present invention; and

FIG. 7 illustrates an embodiment of an apparatus according to the present invention.

### DESCRIPTION OF EMBODIMENTS

The following descriptions depict only example embodiments and are not considered limiting in scope. Any reference herein to the disclosure is not intended to restrict or limit the disclosure to exact features of any one or more of the exemplary embodiments disclosed in the present specification.

Furthermore, the terms first, second, third and the like in the description and in the claims are used for distinguishing between similar elements and not necessarily for describing a sequential or chronological order. The terms are interchangeable under appropriate circumstances and the embodiments of the invention can operate in other sequences than described or illustrated herein.

Furthermore, the various embodiments, although referred to as “preferred” are to be construed as exemplary manners in which the invention may be implemented rather than as limiting the scope of the invention.

The term “subject” will be understood to include an individual in a study or a patient. The term “health practitioner” will be understood to include an individual working at a healthcare facility, such as a doctor, a researcher, a nurse, a caregiver etc. The term “healthcare facility” will be understood to include a hospital, a clinic, a clinical site, a nursing home, and urgent care center, etc. The term “3^{rd} party” will be understood to include a representative of the healthcare facility (e.g., manager, director, researcher, etc.), a sponsor (e.g., in a clinical trial or a hospital), an insurance provider, a contract research organization (CRO), etc. The terms “computing device”, “computer device” and “computer system”, associated with a subject, health practitioner and 3^{rd} party, respectively, refer to a mobile/portable device (e.g., a smartphone, laptop, etc.) or a desktop computer, and said devices and system may be different or of the same type.

The terms “compliance”, “adherence”, and “drop-out” are terms used to describe different levels of engagement or participation in a particular behavior or intervention, such as following a medical treatment, a diet or a study protocol. Compliance refers to the extent to which a subject follows the recommended course of action or treatment as directed by a health practitioner or other authority. Compliance is often measured in terms of taking medication or adhering to a specific treatment plan. Adherence is similar to compliance, but refers to the extent to which a subject follows a recommended course of action or treatment plan in their daily lives, including any behavioral or lifestyle changes that are part of the intervention. Adherence can also include monitoring one's own progress and adjusting the treatment plan accordingly. Drop-out refers to a subject who stops participating in a study, program or treatment before it is completed. Drop-out can happen for various reasons, including lack of motivation, side-effects, logistical difficulties or not seeing the expected benefits. Drop-out rates can be an important indicator of the success of an intervention.

### Customized Subject Messages

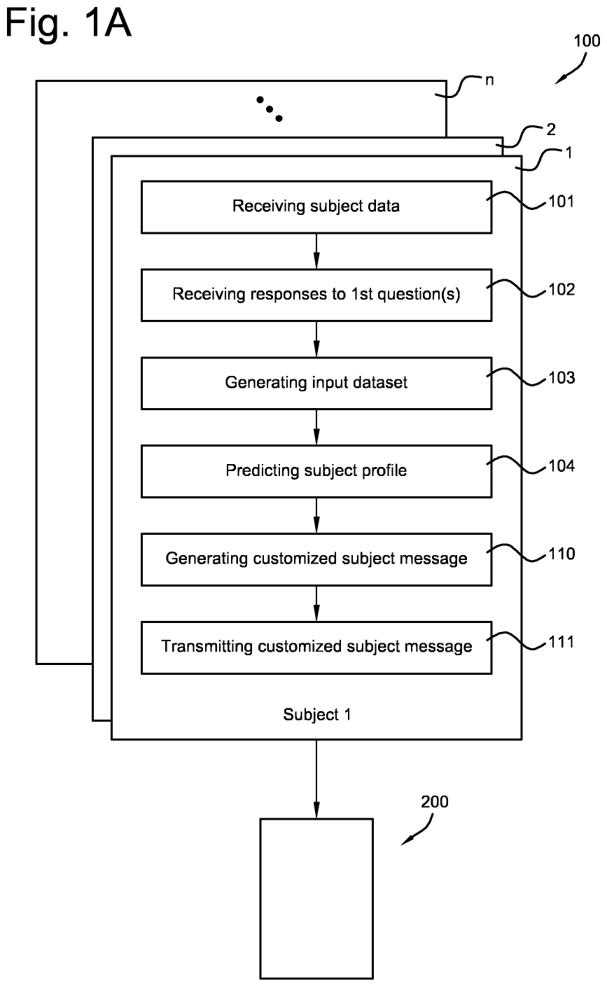

A first aspect of the present invention will be described with reference to FIGS. 1-6. FIGS. 1A and 1B illustrate an embodiment of methods according to the present invention where a server is in communication with a computing device associated with each subject.

In FIG. 1A, the method is shown comprising step 100 including the following steps performed by the server for each subject 1, 2-n in a healthcare facility. For each subject in the healthcare facility, the method comprises step 100 including the steps of receiving 101 (i.e., step 1.a) subject data of the subject; receiving 102 (i.e., step 1.b), from a computing device associated with a given subject, responses of the subject to one or more first questions of a questionnaire, wherein the one or more first questions are determined, by the server, based on the subject data; generating 103 (i.e., step 1.c) an input dataset from the subject data and the received responses; applying 104 (i.e., step 1.d) a trained mathematical model on the generated input dataset to predict a profile of the subject; dynamically generating 110 (i.e., step 1.e) the customized subject message based on the predicted profile of the subject; and transmitting in real-time 111 (i.e., step 1.f) the customized subject message to the computing device associated with the subject.

Although not shown in FIG. 1A, the server may transmit the one or more first questions of the questionnaire to the subject in order to receive the response of step 102. The server may determine the one or more first questions based on the subject data by using a machine learning (ML) model trained on textual data, such as decision tree, a large language model, or collaborative filtering and/or content-based filtering. For instance, the ML model is trained on a dataset comprising historical responses to questions of the questionnaire and historical subject data.

The subject data may include at least one of: location of the subject, demographic data, geographic data, medical data, subject-reported data, and the assigned health practitioner(s). The location of the subject relates to the location where the questionnaire is being taken and/or where the study, research, treatment, diet, etc. is performed on the subject, for e.g., at the healthcare facility, at home, at work, etc. The demographic data may include age, gender, race (or cultural heritage), ethnicity, education level, employment status, etc. The medical data may include medical history, vital signs, laboratory tests, imaging tests, medication records, historical performance of the subject (e.g., compliance, adherence, drop-out, etc.). The subject-reported data may include symptoms, functional status, treatment satisfaction, and/or any previously answered questions, such as those described herein.

In embodiments according to the present invention, step 100 further comprises storing, by the server in a database, at least one of the customized subject message, the subject data and the predicted profile of the subject. The database will be understood as being stored in the memory of the server or in an external memory (e.g., the cloud).

In step 101, the subject data may be received from the healthcare facility or retrieved from storage in the database.

The profile of the subject may include at least one of: personality information, situational information and behavioral information of the subject. The personality information includes information of the subject's personality traits, values, beliefs, attitudes, interests, preferences, etc. The situational information includes information of the subject's response to specific situations or circumstances (e.g., interaction with health practitioner, perception of the health practitioner and/or healthcare facility, perceived burden of the study/treatment/diet, communication preference, etc.). The behavioral information includes information of the subject's behavior including information at least one of compliance, adherence and drop-out. The information on compliance, adherence and drop-out may be in the form of a value, such as score or a probability.

The questions of the questionnaire may relate to one or more of the following:
Average Pain Score (APS)
Functional Index of Hand OsteoArthritis (FIHOA)
Patient Global Assessment
Assessment of Quality of Life (e.g., AQoL or AQoL-4D)
Work Productivity and Activity Impairment (e.g., WPAI-GH)
Global Rating of Change (GRC)
Multidimensional Personality Questionnaire (MPQ or MPS)
Modified Computer Self-Efficacy Scale (CSE)
Missed doses
Etc.

It will be understood that the questions of the questionnaire may relate to one or more of the following:
measuring the impact of the health practitioner's behavior (agreeable, open, severe . . . ) or intervention (oral, acts, etc.),
the sensation of contagion, suggestibility or any other factor likely to influence the balance between deliberate and automatic processing of information on a health symptom onset, evaluation, relief, evolution, etc.,
the level of anxiety, fear, discouragement, hopelessness, depression related to the environment of a healthcare facility or a health practitioner,
evaluating the impact of an individual's environment on his belief of a just world, psychological well-being, psychological quality of life, life satisfaction, resistance to stress and depression,
measuring the subject's expectations with respect to an external stimulus, positive and/or negative outcomes of an intervention or a treatment, and for evaluating their propensity to have a positive or a negative attitude with respect to external factors or health symptoms, specific treatments to relieve health symptoms,
evaluating the attitudinal and emotional response of a subject to external stimuli,
measuring the level of control that the subject believes to have on his life, the level control of external factors or health symptoms on his life such as luck, fate, life events or powerful others (such as e.g. relatives, health professionals, colleagues at work etc.) and for measuring the level of control of powerful others such as relatives or social learning on their attitude to resist, fight or overcome aggressive external factors or health symptoms,
evaluating the (severity) level of health symptoms,
measuring to which extent the subject estimates that health symptoms influence their general physical and psychological condition comprising his body function, activity, mobility, working ability, relations with other people, sleep, life satisfaction, mood, etc., and to which extent the influence of the health symptoms on his general condition evolve with time,
evaluating to which extent the health practitioner estimates that health symptoms influence a subject's general physical and psychological condition comprising their body function, activity, mobility, working ability, relations with other people, sleep, life satisfaction, mood, etc., and to which extent the influence of the health symptoms on their general condition evolve with time,
characterizing the typology and localization of pain, including the painful areas, how the subject translates pain in terms and qualifications such as painful cold, burning, electric shocks, mechanical shocks, tingling, pins, needles, numbness, itching etc., and the physical status of the painful area such as hypoesthesia to touch, hypoesthesia to prick, pain caused or increased by mechanical actions on the body such as brushing, pinching etc.,
the number of doses (e.g., medicament, pills) the subject did not take or has missed

In step 104, the mathematical model or ML model is preferably trained on a dataset comprising information including historical responses to questions of the questionnaire and historical subject data and historical profiles of subjects. The generated input dataset in step 103 is provided to said model to predict a profile of the subject. Said model may be a linear regression model, a decision tree, a recommender system or any other model known in the art.

It is highly preferred that the model and its parameters evolve while receiving new information including new responses, data and subject profiles, wherein the model fits itself on a (continuously) updated dataset including the historical information and the new information. Examples of such a model are collaborative filtering, content-based filtering or a combination of both. In collaborative filtering, the behavior and preferences of subjects with similar profiles is used to predict the profile of individual subjects. This means that the collaborative filtering algorithm analyzes the subject data and the responses to the questions and finds patterns ad similarities with the subject in question. On the other hand, content-based filtering uses the characteristics of the questions to predict the subject profile based on the subject data and the responses to the questions.

In step 110, the server dynamically generates the customized subject message based on the predicted profile of the subject. The customized subject message may include a personalized comment or warning reflective of the predicted profile of the subject and/or may be configured to provide encouragement and/or reassurance and/or incentive to the subject based on the predicted profile of the subject. Additionally, the customized subject message may include recommendations on actions to be taken by the subject (e.g., for better management of treatment or diet) Advantageously, this allows the customized subject message to be more effectively convincing and/or accepted by the subject. This is particularly advantageous when the customized subject message is based on the personality information in the predicted profile of the subject, particularly based on the personality trait of the subject.

In FIG. 1B, the method is shown comprising steps performed by the server for each subject 1, 2-n in a healthcare facility. In addition to the steps described with reference to FIG. 1A, FIG. 1B shows step 100 including the following steps performed by the server. Upon predicting 104 the profile of the subject or upon performing steps 101-104 (i.e., steps 1.a-1.d), determining 105 (i.e., step 2.a) whether the predicted profile of the subject satisfies a predetermined profile requirement, if so, then the steps 110-111 are performed as described with reference to FIG. 1A, otherwise if not, then then the following steps are performed by the server. Determining 106 (i.e., step 2.b) one or more additional questions of the questionnaire based on the predicted profile of the subject, transmitting 107 (i.e., step 2.c), to the computing device, said one or more additional questions, receiving 108 (i.e., step 2.d), from the computing device, responses of the subject to said one or more additional questions, and repeating steps 104-108 (i.e., steps 2.a-2.d) a predetermined number of times or until it is determined that the predicted profile satisfies the predetermined profile requirement, wherein when steps 104-108 (i.e., steps 2.a-2.d) have been repeated said predetermined number of times or when it has been determined that the predicted profile satisfies the predetermined profile requirement, performing steps 110 and 111 (i.e., steps 1.e-1.f).

In step 105, the predetermined profile requirement may be one of the following: that the subject is predicted to be non-compliant, that the subject is predicted to be non-adherent, and that the subject is predicted to drop out. Alternatively, the predetermined profile requirement may be one of the following: that the subject is predicted to be compliant, that the subject is predicted to be adherent, and that the subject is predicted to not drop out. It is highly preferred that the determining whether the predicted profile of the subject satisfies the predetermined profile requirement includes determining whether the prediction (i.e., the predicted profile) has a probability (or confidence rate) greater than a predetermined threshold, such as greater 70%, greater than 75%, greater 80%, greater than 85%, greater than 90%, etc. For example, if the predicted profile of the subject has a probability (or confidence rate) of more than 90% that the subject is non-compliant, then the predetermined profile requirement is satisfied and steps 110 and 111 are performed as described herein.

In step 106, the server may determine the one or more additional questions based on the subject data by using a machine learning (ML) model trained on textual data, such as a decision tree or large language model. For instance, the ML model is trained on a dataset comprising historical responses to questions of the questionnaire and historical subject data. This ML model may be the same model used to determine the one or more first questions.

Steps 105-108 may be repeated until the predetermined profile requirement has been met. In some instances, it would be advantageous to save time and limit the number of repetitions to a predetermined number of times, such as at least 1, at least 2, at least 3, at least 4, at least 5, at least 6, at least 7, etc. The predetermined number of times steps 105-108 (i.e., steps 2.a-2.d) are repeated is based on at least one of: a budget constraint, a confidence rate of the predicted profile exceeding a threshold, and a time limit.

The method may further comprise dynamically updating, by the server, the predicted profile of the subject upon repeating step 104. As described herein, step 100 comprises storing, by the server in a database, predicted profile of the subject. It is preferred that the predicted profile of the subject is stored upon preforming step 104. In embodiments, the server thus dynamically updates the predicted profile in the database upon repeating step 104.

As shown in FIG. 1B, when the predetermined profile requirement has been met, then steps 110 and 11 are performed as described herein.

### Customized Health Practitioner Messages

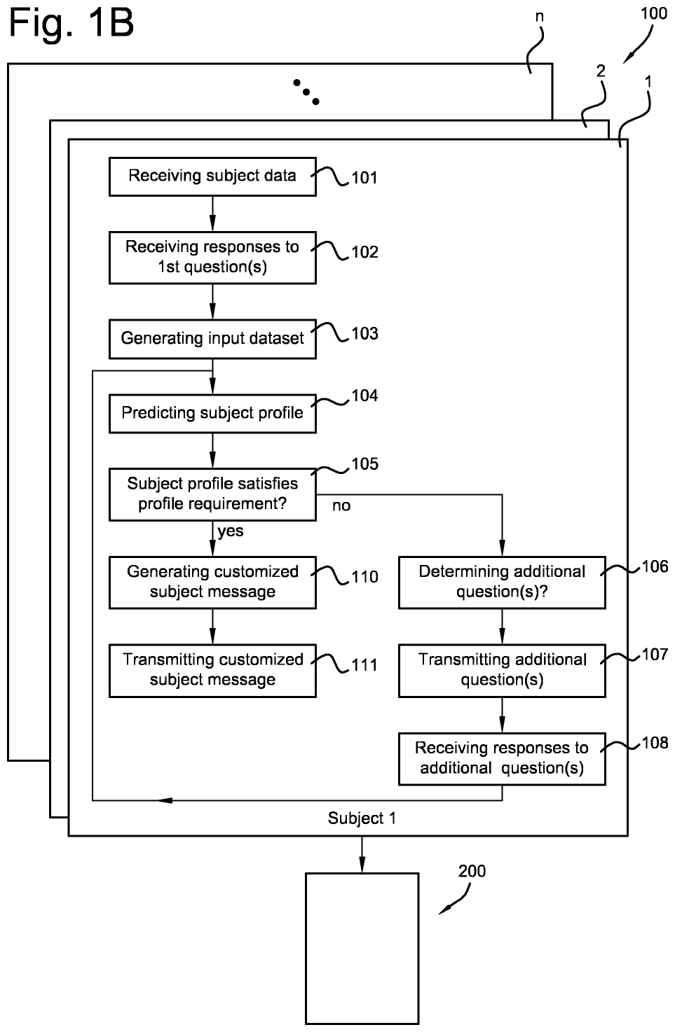

FIG. 2 illustrates an embodiment of a method according to the present invention where a server is in communication with a computer device associated with a health practitioner assigned to a plurality of subjects. It will be appreciated that more than one health practitioner may be assigned the same subject, preferably to the same plurality of subjects.

Step 200 shown in FIG. 2 comprises steps being performed after the steps shown in FIG. 1A or FIG. 1B are performed, preferably after the customized subject message has been transmitted in step 111. It will be appreciated that the steps 200 of FIG. 2 may be performed after step 100 is performed for said at least one of the plurality of subjects, as described herein. Thus, step 100 and step 200 may be performed in parallel, thereby improving the operation of the computer/computing devices/systems in the network environment for the healthcare facility. It will be further appreciated that step 200 may be performed at most for each subject (i.e., performed a number of times equal to the number of subjects in the healthcare facility), and preferably may be performed for at least each health practitioner (i.e., performed a number of times equal to the number of health practitioners in the healthcare facility).

In FIG. 2, the method is shown comprising steps performed by the server as follows. Determining 201 (i.e., step 1.b) that a predetermined configuration requirement has been met; receiving 205 (i.e., step 1.c) healthcare facility data of the healthcare facility; determining 209 (i.e., step 1.d) a performance of at least one of the plurality of subjects based on the healthcare facility data and on the subject data and predicted profile of said at least one of the plurality of subjects; dynamically generating 210 (i.e., step 1.e) the customized health practitioner message based on the determined performance; and transmitting 211 (i.e., step 1.f) in real-time the customized health practitioner message to a computer device associated with the health practitioner assigned to said at least one of the plurality of subjects.

Although not shown in FIG. 2, when it is determined that the predicted profile satisfies the predetermined profile requirement or when steps 105-108 (i.e., steps 2.a-2.d) have been repeated the predetermined number of times, then the method may comprise transmitting in real-time, by the server to the computer device associated with the health practitioner assigned to the subject, the predicted profile of the subject: This ensures that the health practitioner assigned to the subject is informed that the subject has answered the relevant questions and a profile of said subject has been predicted. Furthermore, this allows the health practitioner to learn early on, before they receive a customized health practitioner message, about said particular subject, thereby improving the interaction between the health practitioner and the subject. This is particularly advantageous, when the predicted profile of the subject is transmitted to the health practitioner before step 110 or 111 and after the step of determining 105 that the predetermined profile requirement has been satisfied, thereby allowing the health practitioner to receive the predicted profile without delay. In some instances, the predicted profile may be transmitted after or during step 111 to ensure that the subject is provided enough time to receive the customized subject message and performed the necessary actions before the heath practitioner is notified and contacted/visited the subject.

As shown in FIG. 2, the server determines 201 whether a predetermined configuration requirement has been met. The predetermined configuration requirement may be any of the following. Step 100 (i.e., step 1.a) has been performed for at least one of the plurality of subjects to which the health practitioner has been assigned; a timer has expired since step 100 has been performed for said at least one of the plurality of subjects, particularly since step 111 has been performed; and it has been determined that the predicted profile of said at least one of the plurality of subjects satisfies the predetermined profile requirement. In such cases, step 200 is performed at most for each subject (i.e., performed a number of times equal to the number of subjects in the healthcare facility).

In preferred embodiments, predetermined configuration requirement may be any of the following. Step 100 (i.e., step 1.a) has been performed for the plurality of subjects to which the health practitioner has been assigned; the timer has expired since step 100 has been performed for the plurality of subjects to which the given health practitioner has been assigned; and it has been determined that the predicted profile of the plurality of subjects satisfies the predetermined profile requirement. In such cases, step 200 is performed for at least each health practitioner (i.e., performed a number of times equal to the number of health practitioners in the healthcare facility).

In step 209, the performance information of a subject may include information on factors influencing the responses of the subject and/or the predicted profile of the subject, such as environmental factors or personal factors.

Step 209 may comprise performing by the server statistical analysis to determine the performance information. The statistical analysis may be performed by the server and may include correlation or regression analysis. The predicted profile and/or the subject data of said at least one of the plurality of subjects may be correlated to the healthcare facility data to quantify the associated between the predicted profile and healthcare facility data. Alternatively, regression analysis may be performed to assess the relationship between the subject data, the predicted profile and the healthcare facility data. Thus, an associated or a relationship between one or more variables in the healthcare facility data and one or more variables in at least one of the predicted profile and subject data may be determined to determine the performance information of a subject.

The healthcare facility data may include at least one of: healthcare facility turnover, a number of staff members, an expected number of subjects per month, and a number of health practitioners. Thus, it is advantageous to understand any association or relationship between the subject data and predicted profile of said at least one of the plurality of subjects and any factors or variables in the healthcare facility. This is particularly advantageous the more subjects in the plurality of subjects are analyzed.

The step of dynamically generating 210 the customized health practitioner message may be further based on the predicted profile of said at least one of the plurality of subjects.

In step 210, the customized health practitioner message may include instructions and/or recommendations relating to at least one of: applying a reinforcement procedure for subject engagement relative to the predicted profile of a subject to which the health practitioner is assigned, adapting the interactions or scheduled visits of the health practitioner with the subject to which the health practitioner is assigned (e.g., increasing visits, decreasing visits, more regular visits, more irregular visits), removing a particular subject from the study/treatment, helping the management of treatment or diet of the subject and changing/adapting treatment of the subject. Advantageously, this allows actions taken by the health practitioner to be more effective and suitable for the subject. This is particularly advantageous when the customized health practitioner message is further based on the personality information in the predicted profile of the subject, particularly based on the personality trait of the subject.

Based on the result of the statistical analysis in step 209, the server may determine the performance information of a subject. For example, if there is a high correlation between a variable in the behavioral information in the predicted profile of a subject and the increased healthcare facility turnover, then the performance information is determined to be more environmental, and thus, the predicted profile of the subject is more effectively improved by customizing the health practitioner message to include more frequent visits to improve the relationship between the subject and the health practitioner. In another example, if it is determined that there is a low relationship between the subject data and predicted profile and the healthcare facility data, then the performance information is determined to be more personal than environmental, and thus, the predicted profile of the subject is more effectively improved by customizing the health practitioner message to include reinforcement measures. In even another example, it may be detected which health practitioners assigned to a subject having a high compliance. In that regard, the customized health practitioner message includes recommendations for adjustment of the interactions with the patient to improve compliance, and/or a call for action if the compliance is dropping. Thus, this allows the customized health practitioner message to include more effective instructions which the health practitioner could act upon to improve the performance information of said at least one of the plurality of subjects.

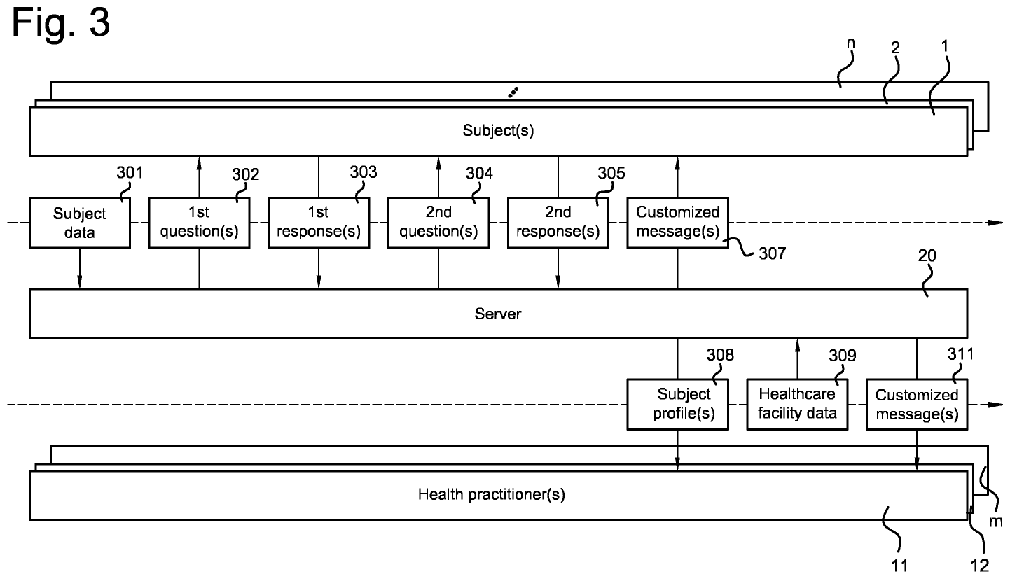

FIG. 4 illustrates an embodiment of a method according to the present invention where a server is in communication with a computer device associated with each health practitioner.

In FIG. 4, the method is shown comprising steps performed by the server for each health practitioner 11, 12-m in a healthcare facility. Said method corresponds to the method described with reference to FIG. 2. Thus, steps 400, 401, 405, 409, 410, 411 respectively correspond to steps 200, 201, 205, 209, 210, 211. Only the differences will be described here for the sake of brevity.

It will be appreciated that the method according to the present invention (e.g., steps 100 and 200) may be repeated for each subject in a predetermined schedule, preferably on a regular basis, such as daily, weekly, monthly or quarterly.

### Customized 3^{rd} Party Message

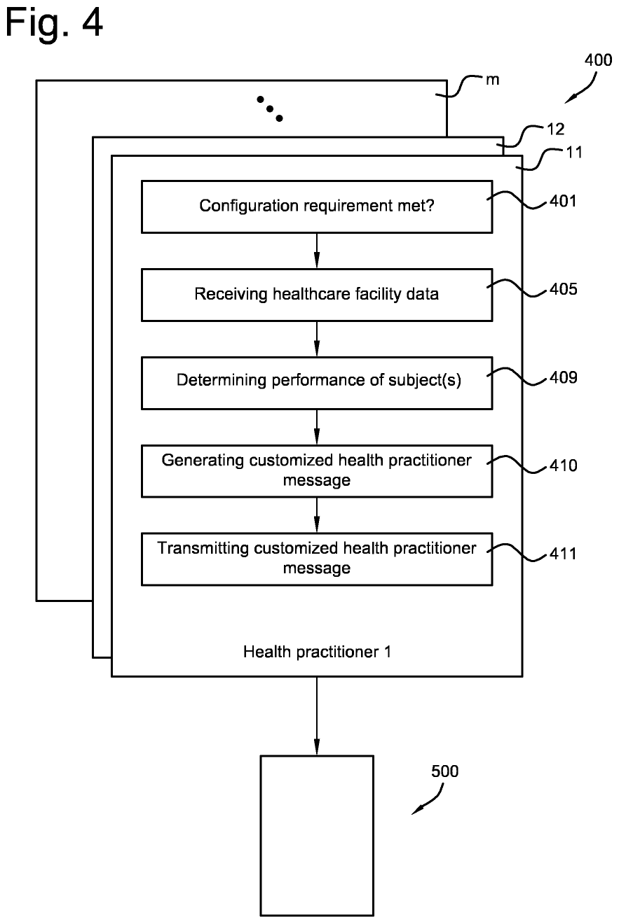

FIG. 5 illustrates an embodiment of a method according to the present invention where a server is in communication with a computing system associated with a 3^{rd} party. In FIG. 5, the method is shown comprising step 500 having steps performed by the server as follows. Determining 509 (i.e., step 9.a) performance information of each subject and/or health practitioner based on the healthcare facility data and on the subject data and predicted profile of each subject; dynamically generating 510 (i.e., step 9.b) a customized 3rd message based on the determined performance information; and transmitting 511 (i.e., step 9.c) in real-time the customized 3^{rd} party message to a computing system associated with the 3^{rd} party.

As will be understood with reference to FIG. 5, the after step 400 is performed by the server for at least one health practitioner, preferably or each health practitioner, step 500 is performed thereafter, preferably after the customized health practitioner message has been transmitted in step 411. It will be appreciated that the steps 500 of FIG. 5 may be performed after step 400 is performed for at least one of the health practitioners. Thus, step 400 and step 500 may be performed in parallel, thereby improving the operation of the computer/computing devices/systems in the network environment for the healthcare facility. It is highly preferred that step 500 is performed after step 400 is performed for each health practitioner and after step 100 is performed for each subject.

As shown in FIG. 5, the method further includes determining 509, by the server, performance information of each subject and/or each health practitioner. The performance information of a subject may include information on factors influencing the responses of the subject and/or the predicted profile of the subject, such as environmental factors or personal factors. The performance information of a health practitioner may include information on factors influencing the actions taken by the health practitioner (e.g., monitoring, intervention, education of subject, support of subject, etc.), such as environmental factors or personal factors.

Step 509 may comprise performing by the server statistical analysis to determine the performance information. The statistical analysis may be performed by the server and may include correlation or regression analysis. The predicted profile and/or the subject data of each subject may be correlated to the healthcare facility data to quantify the associated between the predicted profile and healthcare facility data. Alternatively, regression analysis may be performed to assess the relationship between the subject data, the predicted profile and the healthcare facility data. Thus, an associated or a relationship between one or more variables in the healthcare facility data and one or more variables in at least one of the predicted profile and subject data may be determined to determine the performance information of each subject and/or each health practitioner.

The step of dynamically generating 510 the customized 3^{rd} party message may be further based on the predicted profile of said at least one of the plurality of subjects.

In step 510, the customized 3^{rd} party message may include instructions relating to at least one of: re-assigning the health practitioners to the subjects, adjusting the number of subjects to which a given health practitioner is assigned, adjusting the number of healthcare practitioners, retraining at least one of the health practitioners, and closing the recruitment of subjects in the heathcare facility. Advantageously, this allows actions taken by the health practitioner to be more effective and suitable for the subject. This is particularly advantageous when the customized health practitioner message is further based on the personality information in the predicted profile of the subject, particularly based on the personality trait of the subject.

Based on the result of the statistical analysis in step 509, the server may determine the performance information of each subject and/or each health practitioner. For example, if there is a high correlation between a variable in the behavioral information in the predicted profile of each subject and the health practitioner assigned to the subject, wherein a majority of the plurality of subjects assigned to a given health practitioner have a predicted profile showing different behavioral information towards the given health practitioner than the other subjects to which a different health practitioner is assigned, then the performance information of the plurality of subjects is determined to be more environmental and the performance information of the given health practitioner is determined to be personal, and thus, the predicted profile of the plurality of subjects is more effectively improved by customizing the health practitioner message to include more explicit instructions on how to engage better with a subject, and preferably the customized 3^{rd} party message includes retraining the given health practitioner. In another example, it may be detected for subjects to which only one health practitioner is assigned perform worse (e.g., are less compliant, or more susceptible to drop-out) compared to subjects with more than one health practitioner. In that regard, the customized 3^{rd} party message includes recommendations to reassign the health practitioners, such that each subject is assigned more than one health practitioner and/or a call for action if the compliance is dropping (e.g., reducing the number of subject assigned to a given health practitioner). Thus, this allows the customized 3^{rd} party message to include more effective information to the 3^{rd} party about the status of the healthcare facility and the performance of the subject and/or health practitioners therein.

The customized messages may be provided as a voice recording, but it is preferred that the customized messages are displayed on a graphical user interface (GUI) of the computing device, computer device and computer system associated with the subject, health practitioner and 3^{rd} party, respectively. For example, the customized message may be a video and/or a visualization of text. The present invention is not limited to all customized messages being displayed on the GUI in the same way. It will be appreciated that the way the customized message is displayed on the GUI is based on the subject profile or on the respective statistical analyses. Note that the term “customized messages” includes the customized subject message, the customized health practitioner message, and the customized third party message.

Furthermore, by dynamically generating customized messages and transmitting the same in real-time, the present invention allows to reduce the delay between determining whether a subject would be non-compliant or non-adherent and performing a reinforcing action by at least one of the 3^{rd} party and/or the health practitioner. This reduces the chances that the subject is non-compliant, non-adherent or drops-out, and improves the efficacy of the study or treatment by ensuring that subject who would be non-compliant, non-adherent or drop-out can be re-assigned or replaced early on in the study or the treatment can be re-assigned or adapted early on in the treatment.

It will be further appreciated that the method according to the present invention (e.g., steps 100, 200 and 300) may be repeated for each subject in a predetermined schedule, preferably on a regular basis, such as daily, weekly, monthly or quarterly.

Thus, the statistical analysis as described herein may provide information on the healthcare facility's performance. This can provide the 3^{rd} party with improved decision making for the selection of a strategy and/or for managing the research, clinical trial, study or treatment at the facility and/or for optimizing the risk-based quality management (RBQM) activities.

### Apparatus and System

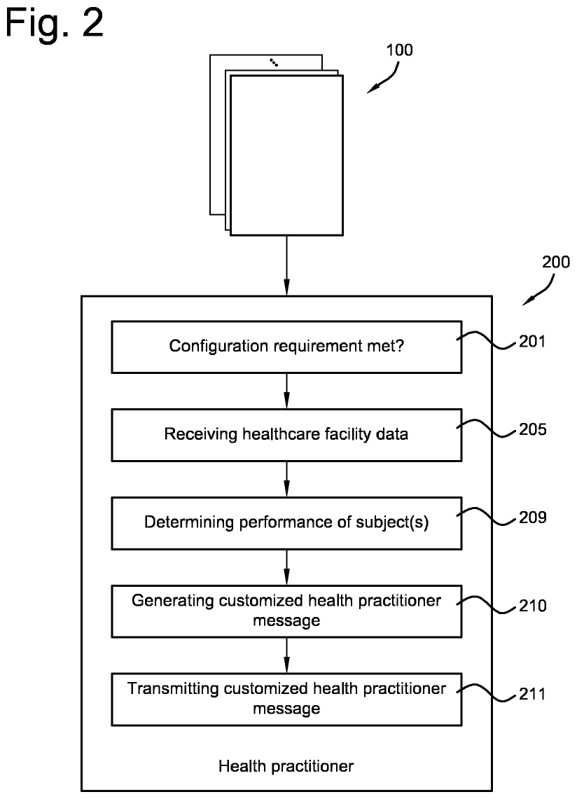

FIG. 3 illustrates an embodiment of a system according to the present invention, where the system comprises the server 20, at least one computing device and at least one computer device. It is preferred that system comprises the server 20, the computing devices associated with the subjects 1, 2-n and the computer devices associated with the health practitioners 11, 12-m.

As shown in FIG. 3, the server 20 receives subject data 301 of a subject, transmits a 1^{st} set of question(s) 302 to the computing device of the subject, receives a 1^{st} set of response(s) 303 from the computing device of the subject, transmits a 2^{nd} set of question(s) 304 to the computing device of the subject, receives a 2^{nd} set of response(s) 305 from the computing device of the subject, transmits a customized subject message to the computing device of the subject. It is preferred that the server 20 communicates with the computing devices of all the subjects 1, 2-n, wherein the server at least transmits customized subject messages to said subject 1, 2-n.

As further shown in FIG. 3, the server 20 receives healthcare facility data 309 and transmits a customized health practitioner message 311 to the computer device of the health practitioner. It is preferred that the server 20 communicates with the computer devices of all the health practitioners 11, 12-m, wherein the server at least transmits customized health practitioner messages to said health practitioners 11, 12-m.

FIG. 3 illustrates a preferred embodiment where the server 20 transmits a (predicted) subject profile to the health practitioner(s) assigned to the subject. It is highly preferred that the server 20 communicates with the computer devices of all the health practitioners 11, 12-m, wherein the server at least transmits the (predicted) subject profiles to the health practitioners 11, 12-m assigned to subjects 1, 2-n.

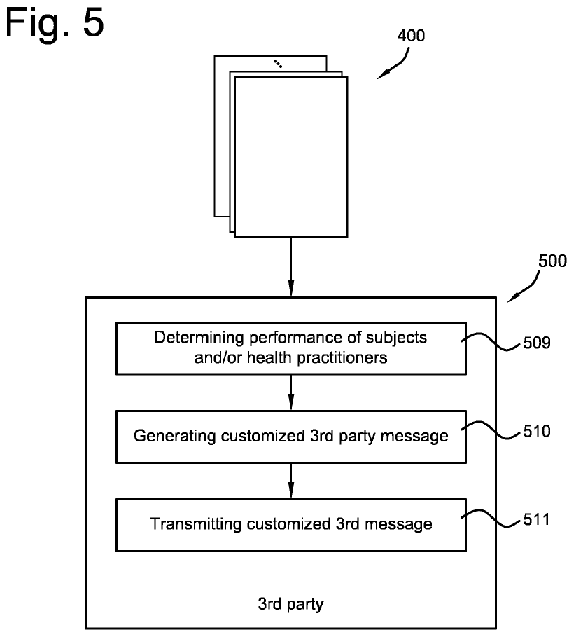

FIG. 6 illustrates an embodiment of a system according to the present invention, where the system comprises the server 20, at least one computer device and a computer system. It is preferred that system comprises the server 20, the computer devices associated with the health practitioners 11, 12-m and the computer system associated with the 3^{rd} party.

As shown in FIG. 6, the server 20 transmits a customized health practitioner message 607 to the computer device of a health practitioner and a customized 3^{rd} party message 611 to the computer system of the 3^{rd} party 30. It is preferred that the server 20 communicates with the computer devices of all the health practitioners 11, 12-m, wherein the server at least transmits customized health practitioner messages to health practitioners 11, 12-m.

The embodiments of FIGS. 3 and 6 may be combined, in that the system may comprise the server 20, the one or more computing devices, the one or more computer devices and the computer system.

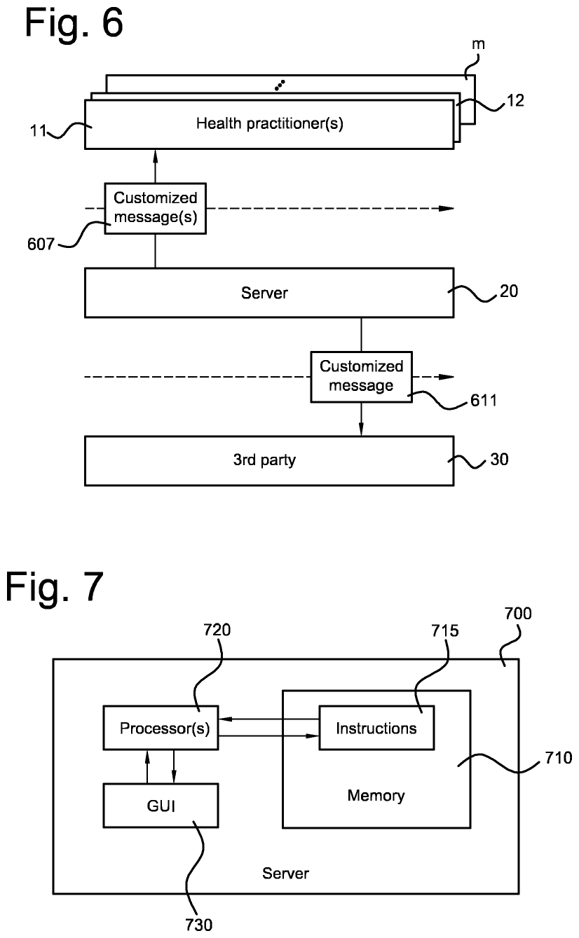

FIG. 7 illustrates an embodiment of an apparatus according to the present invention. The apparatus is the server 700 comprising a non-transitory computer readable medium (e.g., a memory 710) embodying computer executable instructions 715, and at least one processor 720 coupled to the memory 710 and operative by the computer executable instructions 715 to facilitate the method as described above.

The server 700 may further comprise a GUI 730 to which the at least one processor 720 is coupled. The GUI 730 includes one or more inputs (e.g., keyboard, mouse, voice command, touch screen, etc.) and one or more outputs (e.g., display, speaker, etc.). Although not shown, the computing device of each subject, the computer device of each health practitioner and the computer system may comprise a GUI, as described above, including one or more inputs (e.g., keyboard, mouse, voice command, touch screen, etc.) and one or more outputs (e.g., display, speaker, etc.).

The non-transient computer readable medium contains a computer executable software which when executed on a computer system performs the method as defined herein before by the embodiments of the present disclosure. A non-transient computer readable medium may include an electrical connection having one or more wires, a portable computer diskette, a hard disk, a random access memory (RAM), a read-only memory (ROM), an erasable programmable read-only memory (EPROM or flash memory), a portable compact disc read-only memory (CD-ROM), an optical storage device, a magnetic storage device, or any suitable combination of the foregoing. In the context of this document, a non-transient computer readable medium may be any tangible medium that can contain, or store a program for use by or in connection with an instruction execution system, apparatus, device or module.

It will be appreciated that the server 20 may perform any of the steps described above.

1. A computer-implemented method for providing a dynamically customized subject message and a dynamically customized health practitioner message to each subject and health practitioner in a healthcare facility, respectively, wherein each health practitioner is assigned to a plurality of subjects, the method comprising for each subject in the healthcare facility:
a. receiving, by a server, subject data of the subject,
b. receiving, by the server from a computing device associated with a given subject, responses of the subject to one or more first questions of a questionnaire, wherein the one or more first questions are determined, by the server, based on the subject data,
c. generating, by the server, an input dataset from the subject data and the received responses,
d. applying, by the server, a trained mathematical model on the generated input dataset to predict a profile of the subject,
e. dynamically generating, by the server, the customized subject message based on the predicted profile of the subject, and
f. transmitting in real-time, by the server, the customized subject message to the computing device associated with the subject;
g. upon determining, by the server, that a predetermined configuration requirement has been met:
i. receiving, by the server, healthcare facility data of the healthcare facility;
ii. determining, by the server, performance information of at least one of the plurality of subjects based on the healthcare facility data and on the subject data and predicted profile of said at least one of the plurality of subjects;
iii. dynamically generating, by the server, the customized health practitioner message based on the determined performance; and
iv. transmitting in real-time, by the server, the customized health practitioner message to a computer device associated with the health practitioner assigned to said at least one of the plurality of subjects.

2. The computer-implemented method according to claim 1, further comprising:
a. upon performing step 1.d, determining, by the server, whether the predicted profile of the subject satisfies a predetermined profile requirement,
wherein when it is determined that the predicted profile does not satisfy the predetermined profile requirement, the method comprises:
b. determining, by the server, one or more additional questions of the questionnaire based on the predicted profile of the subject,
c. transmitting, by the server to the computing device, said one or more additional questions,
d. receiving, by the server from the computing device, responses of the subject to said one or more additional questions,
e. repeating, by the server, steps 2.a-2.d a predetermined number of times or until it is determined that the predicted profile satisfies the predetermined profile requirement,
wherein when steps 2.a-2.d have been repeated said predetermined number of times or when it has been determined that the predicted profile satisfies the predetermined profile requirement, performing steps 1.e-1.f.

3. The computer-implemented method according to claim 2, wherein when it is determined that the predicted profile satisfies the predetermined profile requirement or when steps 2.a-2.d have been repeated the predetermined number of times, then the method further comprises:
transmitting in real-time, by the server to the computer device associated with the health practitioner assigned to the subject, the predicted profile of the subject.

4. The computer-implemented method according to claim 1, wherein step 1.d comprises performing, by the server, statistical analysis on the healthcare facility data and on the subject data and the predicted profile of the plurality of subjects.

5. The computer-implemented method according to claim 1, wherein the predetermined configuration requirement includes one of:
that step 1.f has been performed for at least one of the plurality of subjects to which a given health practitioner has been assigned;
that a timer has expired since step 1.f has been performed for said at least one of the plurality of subjects; and
that it has been determined that the predicted profile of said at least one of the plurality of subjects satisfies the predetermined profile requirement.

6. The computer-implemented method according to claim 3, further comprising:
displaying a visual representation of the predicted profile of the subject on a graphical user interface (GUI) of the computer device associated with the health practitioner assigned to the subject.

7. The computer-implemented method according to claim 1, further comprising:
displaying the customized subject message on a GUI of the computing device associated with the subject, wherein the customized subject message includes a personalized comment reflective of the predicted profile of the subject and configured to provide encouragement to the subject based on the predicted profile of the subject.

8. The computer-implemented method according to claim 1, further comprising:
displaying the customized health practitioner message on a GUI of the computer device associated with the health practitioner, wherein the customized health practitioner message includes instructions relating to at least one of: applying a reinforcement procedure for subject engagement relative to the predicted profile of a subject to which the health practitioner is assigned, and adapting the interactions of the healthcare practitioner with the subject to which the health practitioner is assigned.

9. The computer-implemented method according to claim 1, further comprising:
a. performing, by the server, statistical analysis on the healthcare facility data, the subject data of each subject and the predicted profile of each subject for determining performance information of each subject and/or health practitioner;
b. dynamically generating, by the server, a customized third party message based on the determined performance information of step 9.a; and
c. transmitting in real-time, by the server, the customized third party message to a computer system associated with a third party.

10. The computer-implemented method according to claim 8, further comprising:
displaying the customized third party message on a GUI of the computer system associated with the third party.

11. The computer-implemented method according to claim 8, wherein the customized third party message includes instructions relating to at least one of: re-assigning the health practitioners to the subjects, adjusting the number of subjects to which a given health practitioner is assigned, adjusting the number of healthcare practitioners, and retraining at least one of the health practitioners.

12. The computer-implemented method according to claim 1, wherein the predicted profile of the subject includes personality information, situational information and behavioral information of the subject.

13. The computer-implemented method according to claim 2, wherein the predetermined profile requirement is one of: that the subject is predicted to be non-compliant, that the subject is predicted to be non-adherent, and that the subject is predicted to drop out.

14. The computer-implemented method according to claim 2, further comprising:
dynamically updating, by the server, the profile of the subject upon repeating step 1.d.

15. The computer-implemented method according to claim 1, wherein the predetermined number of times steps 2.a-2.d are repeated is based on at least one of: a budget constraint, a confidence rate of the predicted profile exceeding a threshold, and a time limit.

16. The computer-implemented method according to claim 1, wherein the mathematical model is trained on a dataset comprising historical responses to questions of the questionnaire and historical subject data and historical profiles of subjects.

17. The computer-implemented method according to claim 1, wherein the healthcare facility data includes at least one of: healthcare facility turnover, a number of staff members, an expected number of subjects per month, and a number of health practitioners.

18. The computer-implemented method according to claim 1, wherein the subject data includes at least one of: location of the subject, demographic data, geographic data, medical data, subject-reported data, and the assigned health practitioner.

19. A non-transitory computer readable medium embodying computer executable instructions which when executed by a computer cause the computer to facilitate a method for providing a dynamically customized subject message and a dynamically customized and health practitioner message to each subject and health practitioner, respectively, in a healthcare facility, wherein each health practitioner is assigned to a plurality of subjects, the method comprising for each subject in the healthcare facility:
a. receiving, by a server, subject data of the subject,
b. receiving, by the server from a computing device associated with a given subject, responses of the subject to one or more first questions of a questionnaire, wherein the one or more first questions are determined, by the server, based on the subject data,
c. generating, by the server, an input dataset from the subject data and the received responses,
d. applying, by the server, a trained mathematical model on the generated input dataset to predict a profile of the subject,
e. dynamically generating, by the server, the customized subject message based on the predicted profile of the subject, and
f. transmitting in real-time, by the server, the customized subject message to the computing device associated with the subject;
g. upon determining, by the server, that a predetermined configuration requirement has been met:
i. receiving, by the server, healthcare facility data of the healthcare facility;
ii. determining, by the server, performance information of at least one of the plurality of subjects based on the healthcare facility data and on the subject data and predicted profile of said at least one of the plurality of subjects;
iii. dynamically generating, by the server, the customized health practitioner message based on the determined performance; and
iv. transmitting in real-time, by the server, the customized health practitioner message to a computer device associated with the health practitioner assigned to said at least one of the plurality of subjects.

20. An apparatus comprising a memory embodying computer executable instructions and at least one processor, coupled to the memory, and operative by the computer executable instructions to facilitate a method for providing a dynamically customized subject message and a dynamically customized and health practitioner message to each subject and health practitioner, respectively, in a healthcare facility, wherein each health practitioner is assigned to a plurality of subjects, the method comprising for each subject in the healthcare facility:
a. receiving, by a server, subject data of the subject,
b. receiving, by the server from a computing device associated with a given subject, responses of the subject to one or more first questions of a questionnaire, wherein the one or more first questions are determined, by the server, based on the subject data,
c. generating, by the server, an input dataset from the subject data and the received responses,
d. applying, by the server, a trained mathematical model on the generated input dataset to predict a profile of the subject,
e. dynamically generating, by the server, the customized subject message based on the predicted profile of the subject, and
f. transmitting in real-time, by the server, the customized subject message to the computing device associated with the subject;
g. upon determining, by the server, that a predetermined configuration requirement has been met:
i. receiving, by the server, healthcare facility data of the healthcare facility;
ii. determining, by the server, performance information of at least one of the plurality of subjects based on the healthcare facility data and on the subject data and predicted profile of said at least one of the plurality of subjects;
iii. dynamically generating, by the server, the customized health practitioner message based on the determined performance; and
iv. transmitting in real-time, by the server, the customized health practitioner message to a computer device associated with the health practitioner assigned to said at least one of the plurality of subjects.

21. The computer-implemented method according to claim 2, wherein the predetermined configuration requirement includes one of:
that step 1.f has been performed for at least one of the plurality of subjects to which a given health practitioner has been assigned;
that a timer has expired since step 1.f has been performed for said at least one of the plurality of subjects; and
that it has been determined that the predicted profile of said at least one of the plurality of subjects satisfies the predetermined profile requirement.

22. The computer-implemented method according to claim 2, wherein the predicted profile of the subject includes personality information, situational information and behavioral information of the subject.
